(19) United States
(12) Patent Application Publication
(10) Pub. No.: US 2009/0154645 A1
(43) Pub. Date: Jun. 18, 2009

(54) TELETHERAPY TREATMENT CENTER

(76) Inventors: Leon LIFSHITZ, Rishon Le'tzion (IL); Michael Marash, Rishon Le'tzion (IL)

Correspondence Address: PEPPER HAMILTON LLP, ONE MELLON CENTER, 50TH FLOOR, 500 GRANT STREET, PITTSBURGH, PA, 15219, US

(21) Appl. No.: 12/185,303

(22) Filed: Aug. 4, 2008

Related U.S. Application Data
(63) Continuation-in-part of application No. 12/127,524, filed on May 27, 2008.
(60) Provisional application No. 60/953,990, filed on Aug. 4, 2007.
(60) Provisional application No. 60/939,923, filed on May 24, 2007.

(51) Int. Cl. A61N 5/10

(52) U.S. Cl. 378/65

(57) ABSTRACT

A method of teletherapy preparation and treatment comprising: providing at least one treatment area comprising a fixed beam irradiation source; providing a plurality of preparation areas; securing a patient to a patient presentation platform in one of the provided preparation areas; rotating and translating the patient, in the one of the provided preparation areas, to a planned irradiation angle and position in relation to the fixed beam irradiation source of the at least one treatment area; transporting the rotated and translated patient from the one of the provided preparation areas to one of the provided at least one treatment area; and irradiating a target tissue of the rotated and translated patient from the fixed beam radiation source at the planned irradiation angle and position. Preferably, the method further comprises imaging the target tissue in the provided treatment area; and finely adjusting the rotation and translation responsive to the imaging.

### PRIORITY AND REFERENCE TO RELATED APPLICATIONS

The present application claims priority to U.S. Provisional Patent Application Ser. No. 60/953,990, bearing the present title, filed on Aug. 4, 2007, which is hereby incorporated by reference. This application is also a continuation in part of U.S. patent application Ser. No. 12/127,524, which claims priority to U.S. Provisional Application Ser. No. 60/939,929, filed on May 27, 2007, which is incorporated herein by reference. This application is furthermore related to U.S. patent application Ser. No. 12/127,391, filed on May 27, 2008, which is also incorporated herein by reference.

### BACKGROUND

The invention relates generally to the field of teletherapy and in particular to a teletherapy treatment center providing separate preparation and treatment areas with inter-area transport at a planned treatment angle.

Teletherapy relates to a treatment methodology in which an irradiation source is at a distance from the body to be treated. X-rays and electron beams have long been used in teletherapy to treat various cancers. Unfortunately, X-rays and electron beams exhibit a linear energy transfer approaching an exponential attenuation function and are therefore of minimal safe use for deeply embedded growths. Recently, the use of heavy particles, particularly hadrons and more particularly protons, in teletherapy has found increasing acceptance due to the ability of heavy particles to penetrate to a specific depth without appreciably harming intervening tissue. In particular, the linear energy transfer of hadrons exhibits an inversed depth profile with a marked Bragg peak at the point where the hadrons deposit most of their energy. The point where the hadrons deposit most of their energy is at the end of the hadron's path. As a result, increased energy can be directed at an embedded growth, unlike X-rays and electron beams, which harm intervening tissues. While the term hadrons include a wide range of particles, protons and various ions are most widely used in therapy. For clarity, this document will describe treatment as being accomplished with protons; however, this is not meant to be limiting in any way.

The charged protons or ions can be focused on a target volume of variable penetration depth. In this way, the dose profile can be matched closely to the target volume with high precision. In one embodiment, in order to ensure complete irradiation of the target growth, a plurality of beams targeted at the embedded growth from several different directions is used. The point at which the plurality of beams intersect, regardless of whether the beams are beamed sequentially or simultaneously, is termed the isocenter. To maximize biological effectiveness of the beams, the isocenter must be precisely collocated with the target growth.

Other prior teletherapy systems use a gantry system carrying a beam generating and delivery system. Unfortunately, the beam generating and delivery system is extremely heavy. Further, the need for such a gantry system, coupled with the prohibitively expensive structure, limits the number of available proton therapy centers. Furthermore, movement of the beam generating and delivery system from one location to another provide delivery of the plurality of beams causes an offset in the isocenter, thus requiring careful readjustment prior to beam delivery. Such a technique is described, for example, in U.S. Pat. No. 6,769,806 issued Aug. 3, 2004 to Moyers.

U.S. Pat. No. 5,851,182 issued Dec. 22, 1998 to Sahadevan, discloses a patient setup and treatment verification system for radiation therapy having diagnostic imaging devices connected to a room containing a megavoltage radiation therapy machine. Daily patient setup for routing and three dimensional conformal radiation therapy and on-line treatment port verification with superimposed isodose are done with a patient on a diagnostic imaging table. The patients are transferred from the diagnostic imaging table to the treatment table without changing the verified treatment position. Such a system is limited to a patient being treated in a supine position, thus requiring a costly gantry based irradiation system.

U.S. Pat. No. 6,730,921 issued May 4, 2004 to Kraft, discloses an ion beam system for irradiating tumor tissues, comprising a first irradiation system having an asymmetrical scanning system of fixed location, which has a central ion beam deflection region for deflection angles up to ±15° with respect to the horizontal direction and a second irradiation system which has an ion beam deflection apparatus for a deflection angle greater than the first irradiation system and a symmetrical scanning unit arrange to pivot synchronously with the deflection angle. The system further discloses preparation rooms, after care rooms, and a plurality of irradiation systems. A patient is secured in a horizontal position on a patient couch in one of the preparation rooms, and this horizontal position is not changed before or during radiation. Such a system is limited to a patient being treated in a supine position, thus requiring the costly second irradiation system and the costly deflection mechanism of the first irradiation system.

Thus, there is thus a long felt need for an improved treatment center layout with improved patient safety and throughput without requiring costly gantries.

### SUMMARY

The present invention provides a treatment center having at least one treatment area with a fixed beam irradiation source and at least one preparation room. Associated with each preparation room is a patient presentation platform arranged to enable presentation of a patient at any position relative to a fixed beam irradiation source.

A patient is secured to the patient presentation platform in one of the preparation rooms. The patient secured to the patient presentation platform is positioned to a treatment position in relation to the fixed beam irradiation source of the treatment area. In one embodiment, the treatment position is determined in accordance with a pre-treatment plan. In another embodiment, the positioning determined realtime, i.e., when the patient is at the treatment center.

In some embodiments, the secured patient is positioned to the treatment position through coarse adjustments. In alternative embodiments, the secured patient is positioned to the treatment position through fine adjustment.

In another embodiment, qualified personnel are provided to supervise the patient and ensure that no ill effects are felt by the patient at the treatment position.

The patient presentation platform is then moved with the patient in the treatment position to the treatment area. In one embodiment, the patient presentation platform is moved under automatic control. In another embodiment, the patient is further imaged at the treatment position in the treatment area and fine adjustments to the treatment position are made in response to the imaging. The patient is then irradiated from the fixed beam irradiation source at the treatment position. In yet another embodiment, the patient is positioned to a second different treatment position in the treatment area. In another embodiment, the patient is imaged at the second treatment position in the treatment area and fine adjustments to the patient position are made in response to the imaging. The patient is then subsequently irradiated. There is no limitation on the number of irradiations at any treatment positions that may be performed. In one embodiment, after irradiation, the patient is returned to the preparation room to be released from the patient presentation platform.

Additional features and advantages of the invention will become apparent from the following drawings and description.

### BRIEF DESCRIPTION OF THE DRAWINGS

For a better understanding of the invention, reference is made, purely by way of example, to the accompanying drawings. It is stressed that the particulars shown are by way of example and for purposes of illustrative discussion of the embodiments of the present invention. In the accompanying drawings:

FIG. 1 illustrates a high level perspective drawing of a treatment center layout in accordance with a principle of the current invention;

FIG. 2A illustrates a high level perspective drawing of an embodiment of a patient presentation platform in accordance with a principle of the subject invention;

FIG. 2B illustrates the patient presentation platform of FIG. 2A positioned in accordance with a principle of the current invention;

FIG. 3A illustrates a detailed drawing of a first translator of the patient presentation platform of FIG. 2 in accordance with a principle of the current invention;

FIG. 3B illustrates a detailed drawing of a second translator of the patient presentation platform of FIG. 2 in accordance with a principle of the invention;

FIG. 3C illustrates a detailed drawing of a third translator of the patient presentation platform of FIG. 2 in accordance with a principle of the invention;

FIG. 4A illustrates a detailed drawing of a first rotator of the patient presentation platform of FIG. 2 in accordance with a principle of the invention;

FIG. 4B illustrates a detailed drawing of a second rotator of the patient presentation platform of FIG. 2 in accordance with a principle of the invention;

FIG. 4C illustrates a detailed drawing of a third rotator of the patient presentation platform of FIG. 2 in accordance with a principle of the invention; and

FIG. 5 illustrates a high level flow chart of a method of teletherapy preparation and treatment in accordance with a principle of the invention.

### DETAILED DESCRIPTION

It is to be understood that the invention is not limited in its application to the details of construction and the arrangement of the components set forth in the description or drawings. The invention is applicable to other embodiments and capable of being practiced in various ways. Also, it is to be understood that the phraseology and terminology employed herein is for the purpose of description and should not be regarded as limiting.

The present embodiments enable a treatment center having at least one treatment area with a fixed beam irradiation source and at least one preparation room. Associated with each preparation room is a patient presentation platform arranged to enable presentation of a patient at any position relative to a fixed beam irradiation source.

The fixed beam irradiation source encompasses a number of irradiation sources. For example, the irradiation source also include scanning and scattering technologies sourced from a fixed location charged hadron source with post beam generation scanning or scattering functionality. Other irradiation sources apparent to one of skill in the art may also be used and fall within the scope of this disclosure.

A patient is secured into the mobile patient presentation platform in one of the preparation rooms. The patient is positioned to a treatment position in relation to the fixed beam irradiation source of the treatment area. In some embodiments, the patient is positioned to a treatment position in relation to the center axis of the fixed beam irradiation source. The patient can be transported thus without substantial movement with respect to the patient support mechanisms.

In one embodiment, the patient is positioned to the treatment position in accordance with a pre-treatment plan. In another embodiment, the treatment position is determined real-time, i.e., while the patient is in the treatment center. Once the treatment position is determined real-time, the patient presentation platform is moved in accordance with the determined treatment position.

In some embodiments, the patient is positioned using course position adjustments. The coarse position adjustment is associated with relatively high-speed motion positioning. For example, the relatively high speed positioning can involve quick translation or rotation of the patient presentation platform, or a combination thereof. In an alternative embodiment, the patient is positioned using fine position adjustments. The fine position adjustment is associated with relatively slow-speed positioning. For example, the relatively slow-speed positioning involves slow, precise translation or rotation of the presentation of the patient presentation platform, or a combination thereof.

In another embodiment, qualified personnel supervise the patient and ensure that no ill effects are felt by the patient during the positioning to the treatment position.

The patient presentation platform is then moved with the patient in the treatment position to the treatment area. In one embodiment, the patient is moved to the treatment area under automatic control. For example, the patient presentation platform may use sensors to guide the patient presentation platform along a path between the preparation room and the treatment area.

In another embodiment, the patient is further imaged at the treatment position in the treatment area and fine adjustments to the treatment position are made in response to the imaging. Alternatively, the patient may be further imaged at the treatment position in the preparation area and fine adjustment to the treatment position. The patient is then moved from the preparation to the treatment area for treatment.

The patient is then irradiated with the fixed beam irradiation source. After the patient is irradiated, in one embodiment, the patient is positioned to a second different treatment position in the treatment area. In another embodiment, the patient is imaged at the second treatment position in the treatment area and fine adjustments are made in response to the imaging. After the imaging and responsive adjustments, the patient is then subsequently irradiated. There is no limitation on the number of irradiations at any presentation position that may be performed.

In one embodiment, the patient is returned to the preparation room after irradiation to be released from the patient presentation platform. In another embodiment, the patient is returned to the same preparation room where the patient was secured to the patient presentation platform.

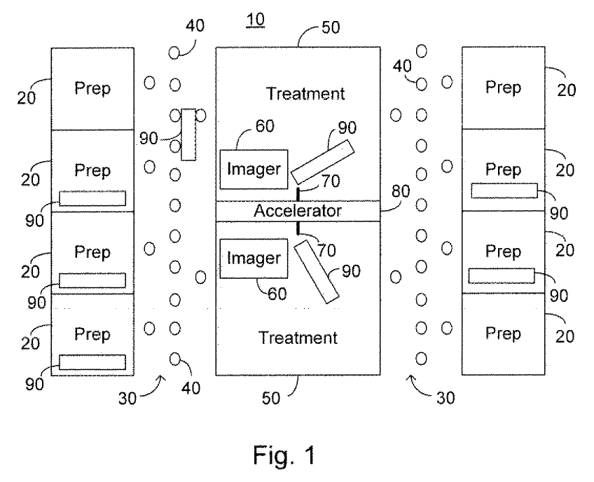

FIG. 1 illustrates a high level perspective drawing of a treatment center 10, in accordance with a principle of the current invention. In one embodiment, each preparation room 20 has an entry/exit to a passageway 30. Further, each treatment area 50 has an entry/exit to the passageway 30. Thus, physical access for each mobile patient presentation platform 90 is provided from each preparation room 20 to each treatment area 50. In another embodiment, the passageway 30 has a plurality of location markers 40 to enable automatic transit of each patient presentation platform 90 between the preparation room 20 and the treatment area 50. In some embodiments, the patient presentation platform uses a sensor to detect the location markers to guide the motion of the patient presentation platform between the preparation room and the treatment area. Other forms automatic transit apparent to one of skill may also be used. Such alternative embodiments fall within the scope of the present disclosure.

In some embodiments, there is no passageway 30. For example, the preparation room may be directly connected to the treatment room. Thus, the mobile patient presentation platform 90 may be moved between the preparation room 20 and the treatment area 50 without having to be moved through a passageway 30. In one embodiment, location markers located between the preparation room and treatment area. The location markers are used to guide the patient presentation platform between the preparation room and the treatment area. In some embodiments, sensors located on the patient presentation platform are used to detect the location markers to control and guide the patient presentation platform between the preparation area and the treatment area.

Accelerator 80 provides hadrons for the fixed beam irradiation source for the treatment area 50. The hadrons from the accelerator exit the fixed beam irradiation source 70 in treatment area 50. In one embodiment, the hadrons exit the fixed beam irradiation source 70 in a pencil shaped beam. In another embodiment, fixed beam irradiation source 70 is arranged to output a generally horizontal beam. In another embodiment, fixed beam irradiation source 70 is arranged to output a generally vertical beam. In yet another embodiment, the generally vertical beam enters from the top of the treatment area, the bottom of the treatment area, or a combination thereof. In yet another embodiment, fixed beam irradiation source 70 is arranged to output a beam at an angle relative to the floor of treatment area 50. Any fixed beam irradiation source arrangement apparent to one of skill in the art may be used without exceeding the scope of this invention.

Imager 60 allows the patient to be imaged while the patient is secured in the patient presentation platform. The imager allows the location of the target tissue to be determined or verified prior to irradiation. In one embodiment, imager 60 is a C-arm computed tomography (CT) imager. In another embodiment, imager 60 may include one of the following: an ultrasound imager, a CT imager, a magnet resonance imager, an x-ray imager, a fluoroscope, a positron emission tomography imager, image guided radiation therapy, and a single photon emission CT imager. The imager may also include a combination of the aforementioned imager types. Other imagers and imaging method apparent to one of skill in the art may also be used without exceeding the scope of the present invention.

In one embodiment, imager 60 may further comprise one or more markers, transmitters, or a combination thereof inserted about or at the target tissue. These markers, transmitters, or a combination thereof are used to identify the areas on the patient to be imaged. In some embodiments, the markers or transmitters may be in cooperative operation with the imager. In another embodiment, imager 60 is arranged to image the patient in the treatment position in relation to fixed beam irradiation source 70 without requiring movement of the patient.

In operation, a patient is secured to a patient presentation platform 90 in one of the preparation rooms 20. The patient is then positioned to a treatment position in relation to the fixed beam radiation source 70. In one embodiment, the positioning is in accordance with a pre-treatment plan. In an alternative embodiment, the positioning is determined in real-time, i.e., while the patient is at the treatment center. Other embodiments to determine the proper treatment positioning apparent to one of skill in the art may also be used.

In some embodiments, the positioning of the patient occurs in the preparation area. In other embodiments, the positioning may occur within the treatment room. In some embodiments, qualified personnel assist in the pre-treatment positioning of the patient. The qualified personnel can supervise the patient and ensure that no ill effects are felt by the patient while the patient is positioned to the treatment position.

In some embodiments, the positioning uses coarse adjustment for the pre-treatment positioning of the patient. The coarse adjustment is associated with relatively high-speed positioning. For example, the high-speed positioning is achieved by quick rotations or translation of the patient presentation platform. In an alternative embodiment, the positioning uses fine adjustment. The fine adjustment is associated with relatively slow-speed positioning. For example, the slow-speed positioning is achieved by slow, precise rotations or translations of the patient presentation platform.

In one embodiment, imaging is performed in the preparation room 20. The imaging is used to make fine adjustments to the treatment position. These fine adjustments increase the accuracy of positioning of the patient's target tissue in relation to the fixed beam irradiation source. In an alternative embodiment, the fine adjustments to the patient's positioning are made in relation to the center axis of the fixed beam irradiation source. In some embodiments, the fine adjustment may occur in preparation room 20. However, the invention is not limited in this respect. For example, the fine adjustment may occur in the treatment area.

With the patient secured on the patient presentation platform at the treatment position, patient presentation platform 90 is then transported to treatment areas 50. In some embodiments, patient presentation platform is transported from the preparation room 20 to the treatment area 50 via passageway 30. In another embodiment, the transportation is accomplished automatically. Specifically, the patient presentation platform 90 uses sensors to detect location markers 40 to guide the patient presentation platform from the preparation room to the treatment area. Alternative embodiments for automatic transportation of the patient from the preparation room to the treatment area apparent to one of skill in the art may also be used. For example, the patient presentation platform may be automatically driven on guide rails connecting the preparation room to the treatment area.

In yet another embodiment, upon arrival of the patient in the treatment area 50, the patient is imaged with imager 60. Fine adjustments to the presentation position are made in response to the image if necessary. In some embodiments, fine adjustments not accomplished in preparation room 20 are accomplished in the treatment area 50.

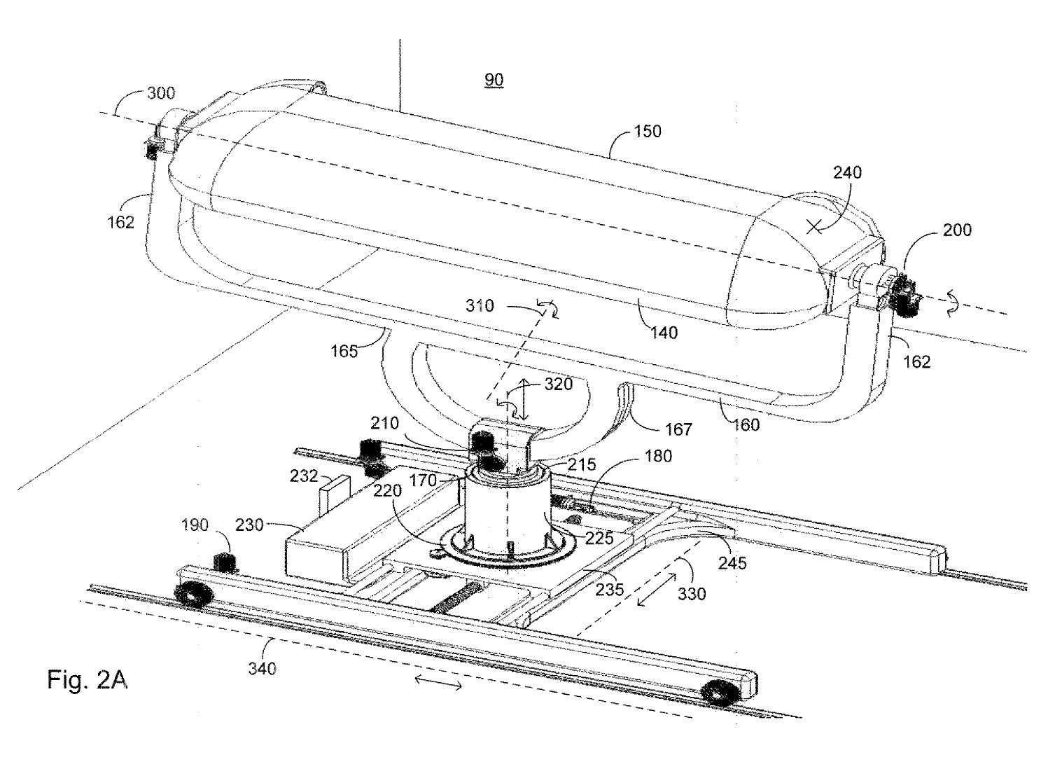

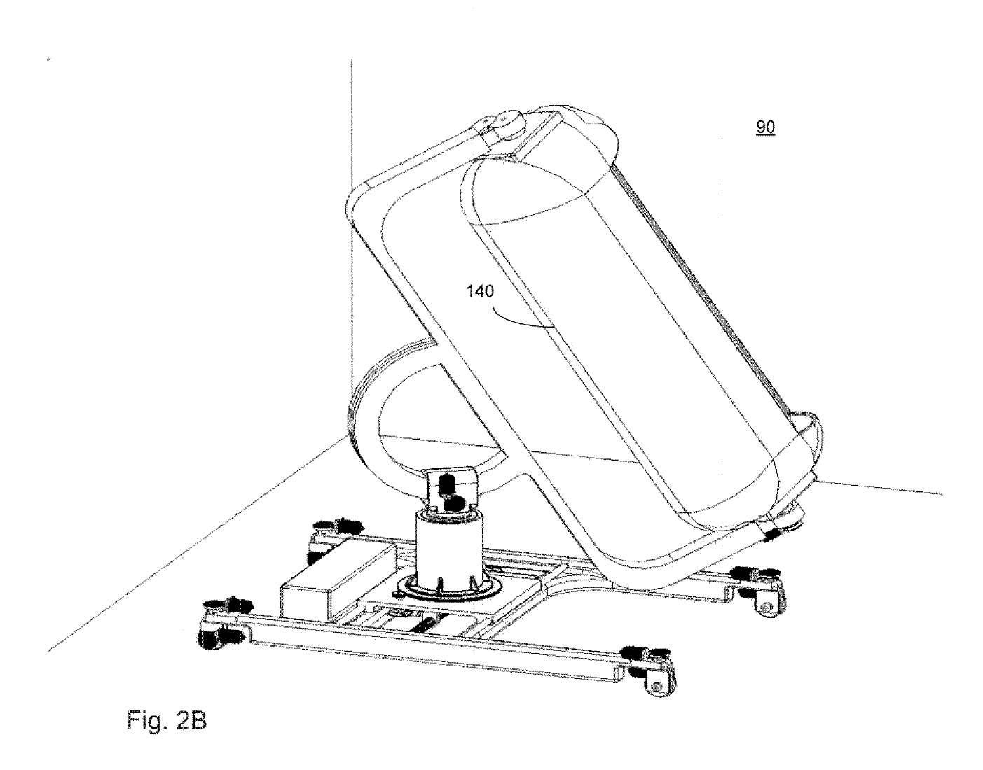

FIG. 2A illustrates a high level perspective drawing of an embodiment of a patient presentation platform 90 in accordance with a principle of the subject invention. The first translator 170, second translator 180 and third translator 190 is arranged to position patient support surface 140. The first rotator 200, second rotator 210 and third rotator 220 is arranged to also position patient support surface 140.

In one embodiment, support fork 160 of the patient presentation platform has a pair of arms 162 and a base 165. The arms 162 of the support fork 160 secures the longitudinal ends of patient support surface 140, while allowing rotation of patient support surface 140 about a first rotation axis 300. The first rotator 200 is coupled with patient support surface 140 about first rotation axis 300. In one embodiment, first rotator 200 provides controlled rotation of patient support surface 140 about first rotation axis 300 of up to 180°. In another embodiment, first rotator 200 provides controlled rotation of patient support surface 140 about first rotation axis 300 of up to 270°. In yet another embodiment, first rotator 200 provides controlled rotation of patient support surface 140 about first rotation axis 300 of up to 360°.

Base 165 of support fork 160 is connected to an arm 167. The arm 167 is coupled with second rotator 210. The patient support surface 140 may also be rotated about a second rotation axis 310. In some embodiments, second rotation axis 310 is orthogonal to first rotation axis 300. Rotation of patient support surface 140 is controlled by second rotator 210. While arm 167 is illustrated as being semicircular, thus limiting the rotation of the patient support surface about axis 310 up to 180°, this is not meant to be limiting in any way. In an alternative embodiment (not shown), arm 167 is about three quarters circular, thus providing rotation about axis 310 up to 270°. In yet another embodiment (not shown), arm 167 is circular and is tangentially connected to base 165, thus providing rotation around axis 310 up to 360°.

In one embodiment, second rotator 210 is connected to one end of axially slidable piston 215. The variably unexposed portion of axially slideable piston 215 is surrounded by a cylinder 225. Axially slideable piston 215 is raised or lowered along a first translation axis and third rotation axis 320 by a hydraulic mechanism.

In one embodiment, cylinder 225 is rotatably connected to table 235. The rotation of cylinder 225 about first translation and third rotation axis 320 is controlled by third rotator 220. In some embodiments, the first translation and third rotation axis 320 is orthogonal to rotation axis 300 and rotation axis 310. In one embodiment, third rotation mechanism 220 provides controlled rotation of patient support surface 140 about third rotation axis 320 of up to 180°. In another embodiment, third rotation mechanism 220 provides controlled rotation of patient support surface 140 about third rotation axis 320 of up to 270°. In yet another embodiment, third rotation mechanism 220 provides controlled rotation of patient support surface 140 about third rotation axis 320 of up to 360°.

In one embodiment, table 235 is translated along second translation axis 330. In some embodiments, second translation axis 330 is orthogonal to first translation and third rotation axis 320. Table 235 is translated by second translator 180. Table 235 is supported by carriage 245, which is translatable along a third translation axis 340. In one embodiment, the third translation axis 340 is orthogonal to first translation and third rotation axis 320 and second translation axis 330. Carriage 245 is translated by third translator 190.

Position sensor 232 is in communication with controller 230. Position sensor 232 provides position information about the position of the patient presentation platform to controller 230. In one embodiment, position sensor works in cooperation with location markers 40. The location markers 40 are used to guide the patient presentation platform between the preparation room and the treatment area.

The first translator 170, second translator 180, third translator 190, first rotator 200, second rotator 210 and third rotator 220 are responsive to control means 230. In one embodiment, the first translator 170, second translator 180, third translator 190, first rotator 200, second rotator 210 and third rotator 220 each comprise a servo drive mechanism to provide both high-speed coarse adjustment of the patient presentation platform and low speed fine adjustment of the patient presentation platform. Further, the servo drive mechanism may comprise a DC servo with internal or external encoders; however, the present invention is not limited in this way. Alternative drive mechanisms apparent to one of skill in the art may also be used. In another embodiment, separate coarse adjustment and fine adjustment mechanisms are supplied for each of first translator 170, second translator 180, third translator 190, first rotator 200, second rotator 210 and third rotator 220. Each coarse adjustment and fine adjustment mechanism is responsive to controller 230. For example, the separate coarse adjustment and fine adjustment mechanisms can comprise of two separate servo drive mechanisms. One servo drive mechanism would provide coarse adjustment while the other servo drive mechanism would provide fine adjustment. Thus, the separate coarse and fine adjustment mechanisms still provide the ability to coarsely or finely adjust the patient's treatment position. Other drive mechanisms apparent to one of skill in the art may be used without exceeding the scope of the present invention.

In operation, patient presentation platform 90 is placed in a neutral loading position in a preparation room 20. In one embodiment, the patient support surface 140 is presented horizontally and unobstructedly to allow the patient to enter with ease. A patient then is placed in a supine position on patient support surface 140, and secured in place by patient securing mechanism 150. In an alternative embodiment, a vertical loading position is used. In yet another embodiment, a loading position forming an angle between the patient support surface and the floor may also be used.

Patient securing mechanism 150 is illustrated as a cover. In one embodiment, the cover is translucent to the beam output by fixed beam irradiation source 70 and to imager 60, however this is not meant to be limiting in any way. For example, the securing mechanism can be a harness. In another embodiment, patient securing mechanism 150 may comprise of a whole body pods, foam cradles, face masks, cranial halos, bite blocks, or a combination thereof. In yet another embodiment, patient securing mechanism comprises of two C-shaped arms that secure to the ends of the patient support surface. The C-shaped arms allow the irradiation beam to treat the patient's target tissue, while not obstructing the path of the irradiation beam. The C-shaped arms are detachably secured, thus allowing the patient to be easily secured to the patient support surface.

The patient is positioned to a position appropriate for irradiation by fixed beam irradiation source 70. Thus, the patient may be positioned for irradiation at any angle between horizontal and vertical. For example, the patient may be placed in a leaning forward position (i.e. post vertical), in a leaning back position (i.e. post horizontal), rotated partially or completely on a side, or rotated past 90 degrees to the side. In one embodiment, the patient may be positioned by translating or rotating the patient presentation platform simultaneously or sequentially.

Once the patient is secured to patient support surface 140 by patient securing mechanism 150, patient support surface 140 is positioned for irradiation of the patient's target tissue. In one embodiment, the patient support surface is positioned in accordance with a pre-treatment plan. In an alternative embodiment, the patient support surface is positioned in real-time, i.e., the patient position is determined while the patient is at the treatment center. In one embodiment, the positioning of patient support surface 140 is controlled by controller 230 which controls the first translator 170, second translator 180, third translator 190, first rotator 200, second rotator 210 and third rotator 220. The patient support surface 140 is thus rotatable about any of three orthogonal axes 300, 310, and 320 and translatable along any of three orthogonal axes 320, 330 and 340.

In one embodiment, patient support surface 140 is rotatable up to 180° about each of three orthogonal axes 300, 310 and 320. In another embodiment, patient support surface 140 is rotatable up to 270° about at least two of three orthogonal axes 300, 310 and 320. In yet another embodiment, patient support surface 140 is rotatable up to 360° about at least two of three orthogonal axes 300, 310 and 320.

In one embodiment, positioning (e.g., rotation and translation) occurs within preparation room 20. In some embodiments, the positioning within the preparation room provides coarse adjustment. The coarse adjustment is associated with relatively high-speed positioning. In an alternative embodiment, the positioning within the preparation room provides fine adjustment. The fine adjustment is associated with relatively slow-speed positioning. In another embodiment, the position occurs under supervision of qualified personnel active to ensure patient comfort and safety.

Position sensor 232 cooperates with location markers 40 and communicates with controller 230. In one embodiment, controller 230 is operative to move patient presentation platform 90 from the respective preparation room 20 to the treatment area 50 by operation of third translator 190. In some embodiments, the patient presentation platform 90 is moved from the preparation room 20 to the treatment area 50 via passageway 30. Movement of the patient presentation platform 90 is guided by location markers 40. In another embodiment, the patient is moved from the preparation room to the treatment room with the patient secured at the treatment position.

In another embodiment, third translator 190 is further provided with an set of controlled wheels or an internal steering mechanism to enable a full range of motion of patient presentation platform 90 between the preparation room 20 to the treatment area 50. In one embodiment, the third translator 190 is provided with brake locks, thus preventing any unintended movement in the position of patient presentation platform along the floor of treatment area 50.

In the event that the pre-treatment planning prescribes a second treatment angle to be irradiated in the same treatment session, patient support surface 140 may be translated along any of the three translation axes 320, 330, 340 and rotated about any of the three rotation axes 300, 310, 320 in accordance with a second irradiation position of the determined treatment in relation to the fixed beam irradiation source 70. In some embodiments, imager 60 is used to image the patient positioned at the second irradiation angle. Responsive to the imaging by imager 60, patient presentation platform 90 may be finely translated along any of three orthogonal translation axes 320, 330, 340 and rotated about any of three orthogonal rotation axes 300, 310, 320. After any positioning, the patient may be again imaged by imager 60 as required. Irradiation from irradiation source 70 at the second irradiation position is then accomplished.

In one embodiment, after the irradiation is completed, controller 230 is operative to move patient presentation platform 90, by operating third translation mechanism 190, from the treatment area 50 to the preparation room 20. In one embodiment, the patient is moved from the treatment area 50 to the preparation room via passageway 30. In some embodiments, the patient presentation platform is guided through the passage way with location markers 40.

In another embodiment, upon arrival in the preparation room 20 patient support surface 140 is positioned by controller 230 to place the patient presentation platform 90 in the neutral loading position. In other embodiments, the presenting patient support surface 140 is horizontal and unobstructed to enable patient's ease of exit. In some embodiments, the preparation room where the patient arrives is the same preparation room where the patient was secured and immobilized.

FIG. 2B illustrates patient presentation platform 90 positioned in accordance with a principle of the current invention. In one embodiment, patient support surface 140 is rotated about three orthogonal axes, wherein the rotating is up to 180° about each of said three orthogonal axes. In another embodiment, the patient support surface is rotated up to 270° about at least two of said three orthogonal axes. In yet another embodiment, the patient support surface is rotated up to 360° about at least two of said three orthogonal axes. In some embodiments, the patient presentation platform 90 is transported from preparation room 20 to treatment area 50 at the irradiation position.

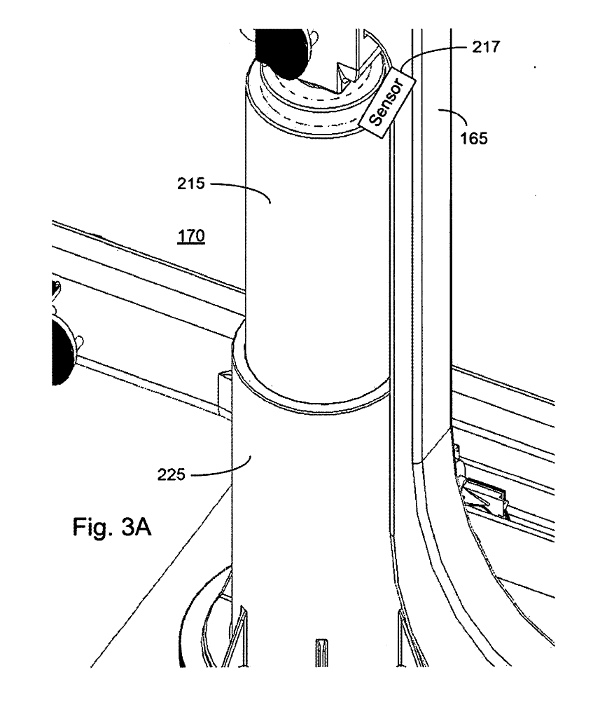

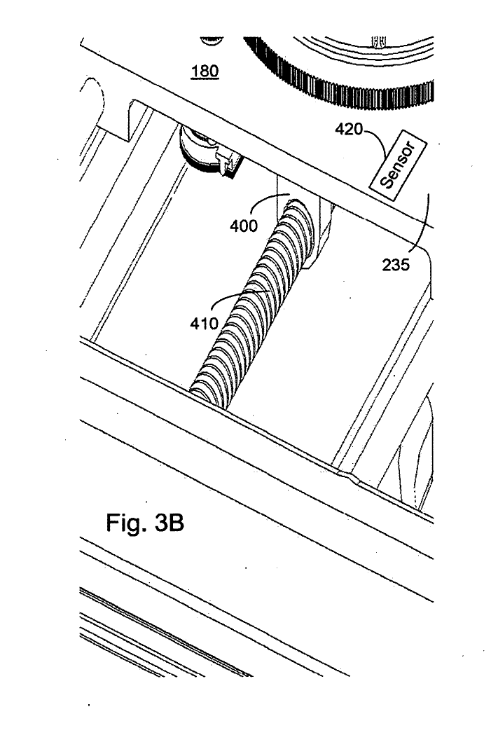

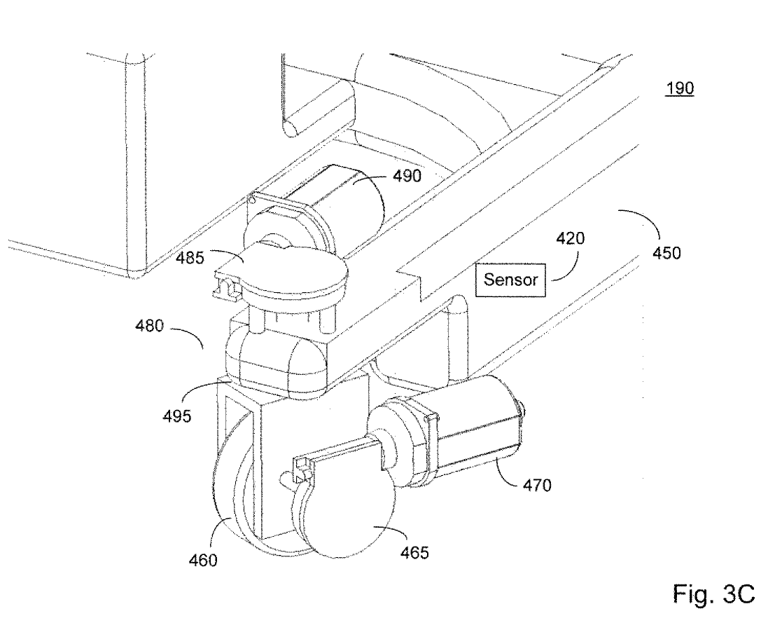

FIG. 3A is a detailed drawing of first translator 170 of patient presentation platform 90, in accordance with a principle of the current invention. FIG. 3A illustrates base 165 of support fork 160 in a vertical position, thus indicating the rotation of patient support surface 140 about second rotation axis 310 to a vertical position. In one embodiment, the first translator comprises an axially slidable piston 215 surrounded by cylinder 225. Axially slideable piston 215 is raised or lowered along first translation axis 320 by a worm gear mechanism (not shown). Other translation mechanisms apparent to one of skill in the art may also be used. The axially slidable piston is responsive to controller 230 of FIG. 2.

The position of the axially slidable position 215 is determined by position sensor 217. In some embodiments, the position sensor may comprise of a mechanical sensor, a piezoelectric sensor, an inductive sensor, an optical sensor, a capacitive sensor, or a combination thereof. In another embodiment, axially slideable piston 215 is raised or lowered along first translation axis 320 by a hydraulic mechanism (not shown) under control of controller 230 of FIG. 2. In other embodiments, axially slideably piston is configured for both coarse and fine control movement. More specifically, in some embodiments, the coarse and fine control movement is achieved by use of servo drive and position sensor 217. The servo drive mechanism may comprise a DC servo with internal or external encoders. In an alternative embodiment, coarse and fine control is accomplished by separate translators under control of controller 230.

FIG. 3B illustrates a detailed drawing of second translator 180 of patient presentation platform 90. In one embodiment, table 235 is secured a threaded receiver 400 configured to receive turnable shaft 410. In some embodiments, turnable shaft 410 is rotatable by a worm gear assembly and servo drive motor (not shown) responsive to control means 230. Other translation mechanisms apparent to one of skill in the art may also be used.

The position of table 235 is determined by position sensor 420 comprising of a mechanical sensor, a piezoelectric sensor, an inductive sensor, an optical sensor, a capacitive sensor, or a combination thereof. In an alternative embodiment, coarse and fine control is accomplished by separate translators under control of controller 230.

FIG. 3C illustrates a detailed picture of third translator 190 of patient presentation platform 90, in accordance with a principle of the invention. In one embodiment, third translator 190 further comprises a steering mechanism for transporting patient presentation platform between areas. The steering mechanism further comprises a outrider arm 450 secured a controlled wheel 460. The controlled wheel 460 is operated by worm gear 465 and rotated by a servo drive motor 470.

In some embodiments, the servo drive motor 470 is controlled by controller 230 and position sensor 420. Position sensor 420 comprises a mechanical sensor, a piezoelectric sensor, an inductive sensor, an optical sensor, a capacitive sensor or a combination thereof. In an alternative embodiment, coarse and fine adjustment control is accomplished by separate translators under control of controller 230. Third translation mechanism 190 also comprises a steering mechanism 480. The steering mechanism 480 consists of a worm gear drive 485 rotated by a servo drive motor 490. Thus, the servo drive motor 490 pivots controlled wheel 460 about pivot 495. Thus, controlled wheel 460 is steerable by steering mechanism 480 to enable controlled motion of patient presentation platform 90 between preparation room 20 and treatment area 50. In an alternative embodiment, coarse and fine control of steering mechanism 480 is accomplished by separate steering mechanisms under control of controller 230.

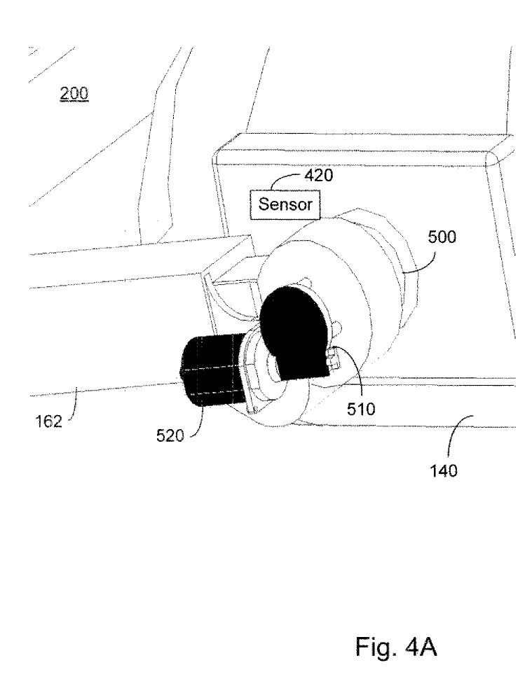

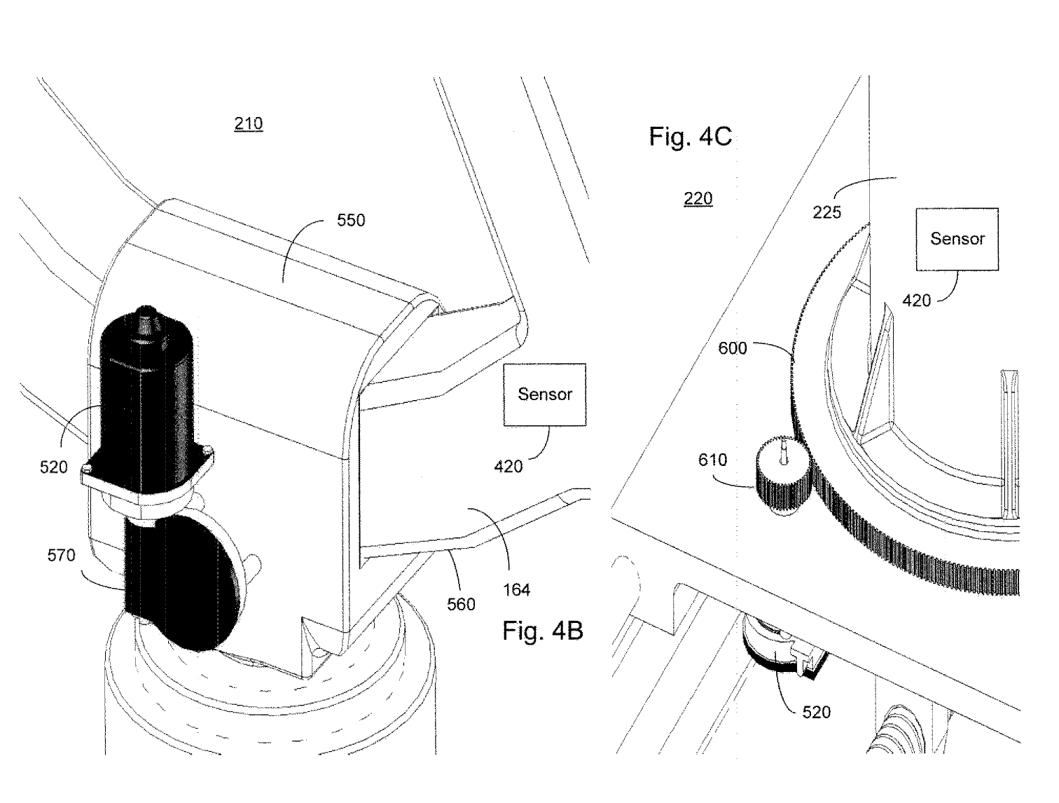

FIG. 4A illustrates a detailed drawing of first rotator 200 of patient presentation platform 90, in accordance with a principle of the invention. In one embodiment, the first rotator 200 comprises an end of one arm 162 of support fork 160 of FIG. 2. The end of one arm 162 is rotatably connected at rotation joint 500 to one end of patient support surface 140. In some embodiments, patient support surface 140 is controllably rotated by worm gear 510 which is connected to a servo drive 520. The servo drive 520 is controlled by controller 230 and position sensor 420. Position sensor is a mechanical sensor, a piezoelectric sensor, an inductive sensor, an optical sensor, a capacitive sensor, or a combination thereof. In an alternative embodiment, coarse and fine control is accomplished by separate rotators under the control of controller 230.

FIG. 4B illustrates a detailed drawing of second rotator 210 of patient presentation platform 90, in accordance with a principle of the invention. In one embodiment, the second rotator comprises arm 167 secured within a holder 550. In some embodiments, arm 167 has a plurality of teeth 560 on an underside of the arm to engage with the matching teeth of a servo drive 570. Patient support surface 140 is controllably rotated by servo drive 570. Servo drive 570 is controlled by controller 230 and position sensor 420. Position sensor may comprise of a mechanical sensor, a piezoelectric sensor, an inductive sensor, an optical sensor, a capacitive sensor, or a combination thereof. In an alternative embodiment, coarse and fine control is accomplished by separate rotators under control of control means 230.

FIG. 4C illustrates a detailed picture of third rotator 220 of patient presentation platform 90, in accordance with a principle of the invention. In one embodiment, the third rotator comprises of cylinder 225 having an external toothed ring 600 secured at one end of cylinder 225. External toothed ring 600 is arranged to engage toothed capstan 610. Servo drive 520 is connected to toothed capstan 610 and controls rotation of toothed capstan 610. Patient support surface 140 is controllably rotated by servo drive 520. Servo drive is responsive to controller 230 and position sensor 420. Position sensor 420 may comprise of a mechanical sensor, a piezoelectric sensor, an inductive sensor, an optical sensor, a capacitive sensor or a combination thereof. In an alternative embodiment, coarse and fine control is accomplished by separate rotators under control of control means 230.

The above has been described in an embodiment in which patient presentation platform 90 comprises an integral mobile support carriage, constituted of outrider arms 450. However this is not meant to be limiting in any way. In another embodiment, a separate transport system is provided to transport patient presentation platform 90, with the patient secured at the planned irradiation angle, from preparation room 20 to treatment area 50.

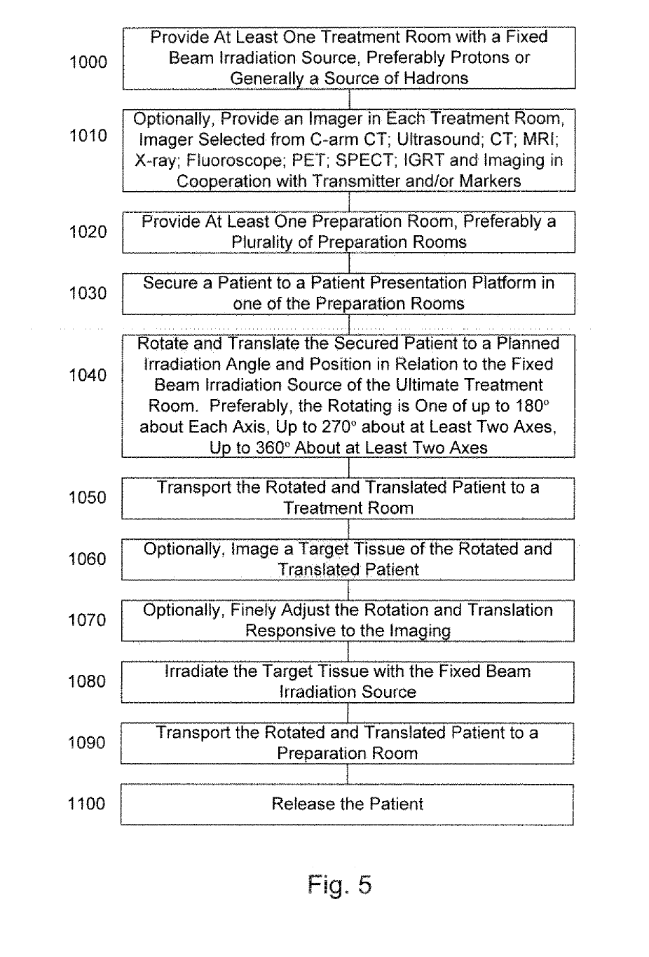

FIG. 5 illustrates a high level flow chart of a method of teletherapy preparation and treatment in accordance with a principle of the invention. In stage 1000, at least one treatment area is provided. The treatment area has a fixed beam irradiation source. In one embodiment, the fixed beam irradiation source is a proton beam. In another embodiment, the fixed beam irradiation source is a hadron source. The fixed beam irradiation source, as used in this document, does not exclude scanning and scattering technologies, which are sourced from a fixed location source with post beam generation scanning or scattering functionality.

In optional stage 1010, an imager is provided associated with each of the provided treatment areas of stage 1000. In one embodiment, the imager is selected from among a C-arm computerized tomography (CT) imager, an ultrasound imager, a CT imager, a magnetic resonance imager, an X-ray imager, a fluoroscope, a positive emission tomography imager; and a single photon emission computed tomography imager. A combination of imaging types may be provided without exceeding the scope of the invention. In another embodiment, the imager may consist of image guided radiation therapy. In yet another embodiment, a C-arm CT imager is used to enable imaging of the patient in a rotated and translated position with respect to the fixed beam irradiation source.

In stage 1020, at least one preparation room is provided. In one embodiment appropriate personnel are provided in each of the preparation rooms. In stage 1030, a patient is secured to a patient presentation platform in one of the preparation rooms of stage 1020. In one embodiment, the patient presentation platform is positioned to a horizontal loading position for ease of patient entry and securing of the patient. In an alternative embodiment, the patient presentation platform is positioned in a vertical loading position.

In stage 1040, the secured patient is positioned to a planned irradiation position in relation to the fixed beam irradiation source of the treatment area. In one embodiment, the patient may be rotated up to 180° about each of three orthogonal rotation axes. In another embodiment, the patient may be rotated up to 270° about at least two of the three orthogonal rotation axes. In yet another embodiment, the patient may be rotated up to 360° about at least two of the three orthogonal rotation axes. In one embodiment, the translation and rotation is accomplished utilizing only coarse adjustment mechanisms. In some embodiments, the patient is imaged, fine adjustments are made in response to the imaging to precisely position the patient in relation to the fixed beam irradiation source.

In stage 1050, the patient secured on the patient presentation platform is transported from the preparation room to a treatment area, and positioned in relation to the fixed beam irradiation source. In some embodiments, the patient is positioned in relation to the center axis of the fixed beam irradiation source.

In optional stage 1060, the target tissue to be irradiated of the positioned patient is imaged with an imager. In one embodiment, a C-arm CT is used, thus enabling the imaging to be accomplished in situ. In optional stage 1070, the positioning of the patient presentation platform is finely adjusted. In some embodiments, the fine adjustment of the patient presentation platform occurs in response to the imaging of the patient. In one embodiment, the translation and rotation adjustment of stage 1070 is accomplished using only fine adjustment mechanisms.

In stage 1080, the target tissue of the patient is irradiated from the fixed beam irradiation source. In the event that additional irradiation positions are prescribed, the patient can be rotated and translated in the treatment area to additional irradiation positions. In some embodiments, imaging and fine adjustment is performed for each irradiation position and irradiation is accomplished in accordance with stage 1080.

After completion of irradiation, in stage 1090 the patient is transported from the treatment area of stage 1050 to a preparation room. In one embodiment, the patient is transported to the preparation room of stage 1030. In stage 1100, the patient is released from the patient presentation platform. In one embodiment, the patient is assisted by the appropriate personnel while being released from the patient presentation platform. In one embodiment, the patient presentation platform is rotated and translated to a horizontal loading/unloading position for ease of exit and release of the patient.

Thus, the present embodiments enable positioning of a patient for irradiation by a fixed beam irradiation source in a preparation room, and moving the patient for irradiation while secured in the position for irradiation.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

The terms “include”, “comprise” and “have” and their conjugates as used herein mean “including but not necessarily limited to”.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

We claim:

1. A method of teletherapy preparation and treatment comprising:
securing a patient to a patient presentation platform in a preparation area;
positioning the patient presentation platform to a treatment position in relation to a fixed beam irradiation source;
transporting the patient presentation platform, with said patient at said treatment position, from the preparation area to a treatment area; and
irradiating a target tissue of the patient with the fixed beam radiation source.

2. The method of claim 1, further comprising:
imaging the target tissue of the patient at the treatment position; and
positioning the patient in response to the imaging of the target tissue.

3. The method of claim 1, wherein the treatment position is determined from a predetermined treatment plan.

4. The method of claim 1, wherein the treatment position is determined when the patient is in the preparation area.

5. The method of claim 1, wherein the treatment position is determined when the patient is in the treatment area.

6. The method of claim 2 wherein imaging of the target tissue is performed by a C-arm computerized tomography imager.

7. The method of claim 2, wherein the imaging is performed by one of the group consisting of ultrasound, computerized tomography, magnet resonance imaging, x-ray imaging, fluoroscopy, positron emission tomography, single photon emission computed tomography, and image guided radiation therapy.

8. The method of claim 2, wherein the imaging is performed in cooperation with markers located about the target tissue.

9. The method of claim 2, wherein the imaging is performed in cooperation with transmitters located about the target tissue.

10. The method of claim 1, wherein said provided fixed beam irradiation source is a proton source.

11. The method of claim 1, wherein the fixed beam irradiation source is a hadron source.

12. The method of claim 1, wherein positioning the patient presentation platform in the preparation area further comprises:
translating the patient presentation platform along any axis of a set of three orthogonal axes; and
rotating said patient presentation platform about any axis of the set of three orthogonal axes.

13. The method of claim 12, wherein the patient presentation platform is rotated up to 180° about any axis of the three orthogonal axes.

14. The method of claim 12, wherein the patient presentation platform is rotated up to 270° about at least two axes of the three orthogonal axes.

15. The method of claim 12, wherein the patient presentation platform is rotated up to 360° about at least two axes of the three orthogonal axes.

16. A treatment center comprising:
a treatment area, the treatment area having a fixed beam irradiation source;
a preparation area; and
a patient presentation platform, the patient presentation platform comprising:
a patient support surface;
a patient securing mechanism, the patient securing mechanism securing a patient to the patient support surface;
a positioner, the positioner coupled to the patient support surface, wherein the positioner positions the patient support surface.
wherein the patient presentation platform is adapted to move between the preparation area and the treatment area without substantial movement of a patient with respect to said support surface during movement of said presentation platform from the preparation area and the treatment area.

17. The treatment center of claim 16, wherein the patient support surface is positioned at a treatment position.

18. The treatment center of claim 17, wherein the treatment position is determined from a predetermined treatment plan.

19. The treatment center of claim 17, wherein the treatment position is determine when the patient is in the treatment center.

20. The treatment center of claim 16, wherein the positioner comprises:
a plurality of rotators, the rotators coupled to the patient presentation platform, wherein the rotators position the patient support surface; and
a plurality of translators, the translators coupled with the patient presentation platform, wherein the translators position the patient support surface.

21. The treatment center of claim 20, wherein the rotators position the patient support surface about any of three orthogonal axes.

22. The treatment center of claim 20, wherein the translators position the patient support surface about any axis of three orthogonal axes.

23. The treatment center of claim 20, wherein the plurality of rotators position the patient support surface up to 180° about any axis of the three orthogonal axes.

24. The treatment center of claim 20, wherein the plurality of rotators position the patient support surface up to 270° about at least two axes of the three orthogonal axes.

25. The treatment center of claim 20, wherein the plurality of rotators position the patient support surface up to 360° about at least two axes of the three orthogonal axes.

26. The treatment center of claim 16, wherein the patient securing mechanism secures a patient to the patient support surface without allowing for patient movement during positioning of the patient support surface.

27. The treatment center of claim 16, further comprising:
a passageway connecting the preparation area and the treatment area, wherein the passageway, the treatment area, and the preparation area each have a location marker, wherein the location marker is used to guide the patient position platform between the preparation area and treatment area.

28. The treatment center of claim 27, further comprising:
a controller in communication with the patient presentation platform to control the position of the patient presentation platform.

29. The treatment center of claim 16, wherein the treatment area further comprises an imager.

30. The treatment center of claim 29, wherein the imager is operative to perform one of a group consisting of ultrasound, computerized tomography, magnet resonance imaging, x-ray imaging, fluoroscopy, positron emission tomography, single photon emission computed tomography, and image guided radiation therapy.

31. The treatment center of claim 29, wherein the imager is operative with markers inserted about the target tissue.

32. The treatment center of claim 29, wherein the imager a C-arm computerized tomography imager.

33. The treatment center of claim 16, wherein fixed beam irradiation source is a proton source.

34. The treatment center of claim 16, wherein fixed beam irradiation source is a hadron source.
